(19) United States
(12) Patent Application Publication
(10) Pub. No.: US 2021/0236412 A1
(43) Pub. Date: Aug. 5, 2021

(54) COSMETIC COMPOSITION OF NON-AQUEOUS THIXOTROPIC NAIL VARNISH CONTAINING A NAIL-HARDENING AGENT, USE AND IMPLEMENTATION METHOD

(71) Applicant: FIABILA, MAINTENON (FR)

(72) Inventor: Olivier NOUGUEREDE, HANCHES (FR)

(21) Appl. No.: 17/049,110

(22) PCT Filed: Apr. 24, 2019
(86) PCT No.: PCT/FR2019/050966

§ 371 Date: Oct. 20, 2020

(30) Foreign Application Priority Data
Apr. 25, 2018 (FR) 18 53597

(51) Int. Cl. A61K 8/96; A61K 8/73; A61K 8/36; A61K 8/24; A61Q 3/02

(52) U.S. Cl. CPC A61K 8/965; A61K 8/731; A61Q 3/02; A61K 8/24; A61K 8/36

(57) ABSTRACT

The invention concerns a cosmetic composition of non-aqueous thixotropic nail varnish containing a film-forming agent comprising nitrocellulose, at least one organic solvent, and a thixotropic agent, characterised in that it contains sorbic acid or one of the salts of same at a concentration of less than or equal to 1% by weight of sorbic acid and in that the thixotropic agent, present at a concentration of between 0.1 and 4% by weight of the composition, is a clay modified with a quaternary amine, neutralised by a strong acid at a concentration of less than 0.1% by weight. The sorbic acid, or one of the salts of same, is used as a hardening agent in such a cosmetic composition applied to nails free of fungal infection. The invention also concerns a method for strengthening and hardening nails free of fungal infection, in particular cracked, thin, brittle, soft or split nails, consisting of applying a composition containing sorbic acid or one of the salts of same topically to said nails and/or the periphery of same.

### FIELD OF THE INVENTION

The invention relates to the field of cosmetic compositions for the nails, more particularly of cosmetic compositions intended to strengthen and harden the nails, and also to the use of a nail-hardening agent, and to the associated method.

### STATE OF THE ART

It is well known that the nails, which are composed of keratin, are sometimes weakened, brittle, split or cracked. In addition to the deterioration in the esthetic appearance of these nails, this results in significant inconvenience for a person in their daily life, mainly when these defects concern the nails of the hand: getting caught in textiles or clothes, or the worsening of the cracks each time something is grasped.

To date, various solutions have been provided for this problem, but these solutions are either imperfect or generate other disadvantages. Mention may thus be made of:

The use of formaldehyde, in proportions which may range up to 5% by weight in the cosmetic composition, has been known for a long time. However, this compound is now subject to very close monitoring on account of its toxic effects on the body, with proven carcinogenic effects. This molecule is also a potent allergen.

The use of glyoxal, a dialdehyde, was described in the patent GB 2 196 978 from 1986. Nevertheless, this product is harmful on inhalation, irritating to the skin and eyes, and may cause sensitization of the skin.

Citral (in the form of a mixture of its two isomers geranial and neral), described in the patent EP 1 408 917, is of course also recognized for its nail-hardening action, but can cause sensitization on contact with the skin and is classified among the identified allergens (according to the list of allergenic substances mentioned in the 7^{th} amendment to European Directive 76/768/EEC). It is therefore used less and less in cosmetics. In addition, these molecules contain a double bond, which makes citral sensitive to UV, and hence subject to yellowing.

The patent EP 2 413 887 has proposed the use, as nail-hardening agent, of a monoaldehyde of formula (I): R—CHO, R being a linear C₅-C₁₂ alkyl radical, or a C₅-C₁₂ alkenyl having a double bond which is not conjugated with the aldehyde —CHO group, not belonging to the list of allergenic substances cited in the 7^{th} amendment to European Directive 76/768/EEC. However, these aldehydes, such as hexanal, have a strong odor, in particular in the varnish in liquid form, which limits their use and their concentration within said cosmetic compositions for the nails.

This list of disadvantages relating to the aldehydes employed in cosmetic compositions for the nails has prompted those skilled in the art to seek other types of non-aldehyde molecules for hardening and strengthening the robustness of human nails.

Among the other nail hardeners, the document U.S. Pat. No. 4,919,920 describes the incorporation of iodine or fluorine in the form of ions into an aqueous cosmetic composition devoid of organic solvent, possibly in the presence of a polyol such as glycerol. Such a use is not appropriate for nail varnishes which comprise a significant phase of organic solvents. Iodine can also trigger allergies in certain users.

There therefore remains a real need to find a compound for hardening the nails which overcomes the aforementioned disadvantages and which can be employed in varied cosmetic compositions, such as varnish, varnish base coat, or peelable varnish compositions, for example.

### AIMS OF THE INVENTION

A first aim of the invention is therefore that of proposing a nail varnish composition incorporating a compound intended to strengthen the structure of the keratin of the nail by hardening it, which does not exhibit the disadvantages mentioned hereinabove.

Another aim of the invention is that of proposing a compound which is not susceptible to aging, and in particular to yellowing, at the concentrations at which it is effective as a hardener for the nails, in a cosmetic varnish composition.

Yet another aim of the invention is that of proposing a hardening compound for the nails which does not exhibit any odor troublesome to the user, at the concentrations at which it is effective as a hardener for the nails, in a cosmetic varnish composition.

Another aim of the invention is that of proposing a thixotropic nail varnish composition, that is to say one which is fluid during the application thereof but which has a greater viscosity at rest, incorporating a hardening compound for the nails which does not alter the thixotropic properties of said varnish.

### DESCRIPTION OF THE INVENTION

To this end, the present invention proposes a nonaqueous thixotropic cosmetic nail varnish composition containing a film-forming agent comprising nitrocellulose, at least one organic solvent, and a thixotropic agent, characterized in that said composition contains sorbic acid or one of its salts at a concentration of less than or equal to 1% by weight and in that the thixotropic agent, present at a concentration of between 0.1% and 4% by weight of the composition, is a clay modified with a quaternary amine, neutralized by a strong acid at a concentration of less than 0.1% by weight of the composition.

The varnish composition is of the solvent type, that is to say based predominantly on (containing more than 50% by weight of) organic solvents. Nonaqueous, or essentially nonaqueous, is understood here to mean a varnish composition, the water content of which does not exceed 3% by weight, preferably less than or equal to 2% by weight, and more preferably less than or equal to 1%.

Sorbic acid is not a toxic product, nor is it allergenic: in fact this molecule is frequently used as a preservative (E200) in the food processing industry, as an antimicrobial agent for inactivating or inhibiting the growth of molds and bacteria in food compositions containing a significant aqueous fraction.

In addition, this molecule, in spite of the presence of two double bonds, does not generate any yellowing over time or any odor disagreeable to the user at concentrations of less than or equal to 1% by weight. Thus, sorbic acid can be used in a cosmetic composition for the nails. In addition, sorbic acid and its salts have the advantage of not posing a risk to the health (sensitization, allergy, toxicity, nor any carcinogenic effect, in contrast to certain active agents of the prior art).

Advantageously, said cosmetic composition contains a concentration of sorbic acid or one of its salts of less than or equal to 0.5% by weight. More advantageously, the cosmetic composition contains a concentration of sorbic acid or one of its salts of between 0.01% and 0.5% by weight, more preferably of between 0.05% and 0.1% by weight.

The thixotropic agent of the varnish composition according to the invention is a clay modified with a quaternary amine, the clay preferably being chosen from bentonite or hectorite. However, these clays comprising quaternary amine grafts are particularly sensitive to the presence of acids, for this reason altering their thixotropic behavior. However, it has been found that, even in the free acid form, sorbic acid did not have any detrimental effect either on the stability of the varnish composition or on its thixotropic character.

The concentration of thixotropic agent is advantageously between 0.5% and 3% by weight, preferably between 1% and 2% by weight, of the composition.

The strong acid which neutralizes the clay modified with a quaternary amine is advantageously phosphoric acid, preferably at a concentration of less than or equal to 0.05% by weight, preferably of less than or equal to 0.03% by weight, of the composition. At such concentrations, phosphoric acid does not discolor the pigments that may be present in the varnish composition while making possible the thixotropic action of the clay.

Advantageously, the concentration of nitrocellulose is between 8% and 30% by weight, preferably between 10% and 25% by weight, more preferably between 12% and 20% by weight, of the composition. The other cellulose derivatives are ruled out as film-forming agent due to their poor adhesion to the nail.

The cosmetic composition according to the invention may be a cosmetic composition in the form of a colored or colorless nail varnish, of a varnish base coat, or of a peelable varnish. It is of course possible to incorporate dyes, pigments and/or pearlescent agents into these cosmetic compositions in order to produce nail varnishes having different colors and appearances.

The inventors have found, surprisingly, that the sorbic acid present in such a thixotropic nail varnish composition confers strengthening and hardening properties on said nails. In the cosmetic nail varnish composition according to the invention, the sorbic acid or one of its salts is thus advantageously used as a hardening agent for the nails.

This use is nontherapeutic, certain organic acids, or their salts, being already known (in particular from the document US2008/0193508) as antifungal agents, in particular against fungal nail infections, that is to say for a therapeutic use.

The present invention therefore also relates to the use of sorbic acid, or of one of its salts, as a hardening agent for the nails in a cosmetic composition as described hereinabove applied to nails free from fungal infection.

The composition may contain a concentration of sorbic acid or of one of its salts of less than or equal to 0.5% by weight, and advantageously a concentration of sorbic acid or of one of its salts of between 0.01% at 0.5% by weight, more preferably of between 0.05% and 0.1% by weight.

Indeed, it has also been noted that the hardening properties of sorbic acid on the nails are present even at very low concentrations of less than or equal to 0.1% by weight (see examples 1-2 hereinbelow).

According to the present invention, sorbic acid as a hardening agent for the nails is incorporated in a film-forming cosmetic composition, such as a colored or colorless nail varnish, a varnish base coat, or a peelable varnish. Film-forming cosmetic compositions, such as varnishes, are preferred as they can remain on the nail for a long time.

Sorbic acid applied to healthy nails, that is to say nails free from fungal infection, may nevertheless be active on nail defects at multiple levels: cracked nails, thin nails, brittle nails, split nails, hardness of the nail, weakness of the nail, without triggering allergic reactions.

The present invention also relates to a method for strengthening and hardening nails free from fungal infection, in particular cracked, thin, brittle, soft or split nails, consisting in topically applying to said nails a cosmetic nail varnish composition as described hereinabove, containing sorbic acid or one of its salts.

The following examples make it possible to illustrate the present invention in a nonlimiting manner. In all the formulations, the concentration of the different constituents is expressed in % by weight of the total weight of the cosmetic composition.

### EXAMPLES

Various cosmetic compositions in the form of nail varnish of the organic-solvent-based varnish type were prepared and applied to nails. The tests hereinafter were performed on these varnishes:
Gloss: 100 μm of varnish was applied to a Leneta-type panel. After drying, the gloss was measured at an angle of incidence of 60° using a Minolta 268 glossmeter.
Flexibility: An aluminum plate covered with a varnish of 300 μm wet is subjected to a slow stamping and the depth of the penetration is measured (ISO1520).
Varnish hardness: It was measured using a Persoz pendulum on a glass plate covered with a varnish with a thickness of 100 μm wet, after drying at room temperature, according to the ISO1522 standard.
Adhesion: A “cross hatch test” is carried out on a glass plate. A score of 0 corresponds to an absence of loss of adhesion. A score of 5 corresponds to a total loss of adhesion.
Solids content: Between 0.5 and 1 g of varnish is poured into a dish. This dish is then placed into an oven for 3 hours at 100° C., then weighed to calculate the solids content of the varnish.
Yellowing stability: The varnishes are packaged in glass bottles and stored in an oven at 50° C. for 1 month. The color of the varnish is then compared to a reference varnish not containing any hardening agent.
Sedimentation stability: A tinted varnish composition containing pearlescent agent and a coloring solution is prepared and is placed in an oven for 1 month at 45° C. A visual observation is made to check whether the pearlescent agent has or has not sedimented out.
Shade stability: A tinted varnish containing a mixture of the following pigments: titanium dioxide, Red 34 and ferric ferrocyanide blue, is placed in an oven at 45° C. for 30 days. The stability of this shade, and more specifically the appearance or not of any syneresis, is observed.
Odor: The odor of the varnish is compared to that of a varnish not containing any hardening agent.
Viscosity: The viscosity of the product is measured at 25° C. using a Brookfield viscometer. The measurements are taken after rotating for one minute at 6 rpm, then after one minute at 60 rpm, and lastly after one minute at 6 rpm. The values are given in mPa.s.

### Examples 1 to 3, Comparative Example 4

Cosmetic compositions in the form of nail varnish of the organic-solvent-based varnish type were prepared from the constituents presented in the following table 1. These compositions contained variable concentrations of sorbic acid, between 0.05% and 1% by weight.

TABLE 1

| | | | | Comp. |
|---|---|---|---|---|
| | Ex. 1 | Ex. 2 | Ex. 3 | ex. 4 |
| Composition | | | | |
| Butyl acetate | 42.52 | 42.47 | 42.07 | 41.57 |
| Ethyl acetate | 20.6 | 20.6 | 20.6 | 20.6 |
| Nitrocellulose | 14 | 14 | 14 | 14 |
| Polyester resin | 9.5 | 9.5 | 9.5 | 9.5 |
| Tributyl acetylcitrate | 6 | 6 | 6 | 6 |
| Isopropyl alcohol | 6 | 6 | 6 | 6 |
| Stearalkonium bentonite | 1.3 | 1.3 | 1.3 | 1.3 |
| Phosphoric acid | 0.03 | 0.03 | 0.03 | 0.03 |
| Sorbic acid | 0.05 | 0.1 | 0.5 | 1 |
| Results | | | | |
| Stability | OK | OK | OK | Yellowing |
| Varnish hardness (s) | 240 | 248 | 243 | 235 |
| Viscosity (mPa · s) | | | | |
| 6 rpm | 3300 | 3250 | 3290 | 3320 |
| 60 rpm | 1030 | 1020 | 1040 | 1030 |
| 6 rpm | 2100 | 2090 | 2120 | 2140 |
| Shade stability | No | No | No | No |
| | syneresis | syneresis | syneresis | syneresis |
| Adhesion | 0 | 0 | 0 | 0 |

The polyester resin of table 1 above is an adipic acid/neopentyl glycol/trimellitic anhydride copolymer diluted to 70% solids content in butyl acetate. The nitrocellulose is wetted to 70% in isopropanol.

These cosmetic compositions are in the form of a varnish, which is colorless and transparent after application to the nail, having a solids content equal to 24% by weight to (measured at 100° C.). One or more coloring substances chosen from pigments, soluble dyes and decorative particles, such as pearlescent agents and glitter, may be added.

It is noted that the maximum amount of sorbic acid that can be incorporated into the varnish without causing yellowing is less than 1% by weight.

In addition, sorbic acid at these concentrations of less than 1% by weight does not exhibit an odor unpleasant to the user. It also does not have any influence on the viscosity of the varnish composition or on the stability thereof.

### Comparative Examples 5-6 and 9, and Example 7

### In Vivo Tests

Four varnish compositions according to example 1, each containing 0.05% by weight of hardening agent, and a reference varnish composition without hardening agent were each subjected to in vivo tests on panels of 30 persons aged at least 20 years for three weeks in order to evaluate the efficacy and the tolerance thereof. None of the persons tested exhibited any lesion at the site of the target zones (here the periphery of the nails of the hand), or any dermatological condition, or any fungal infection of the nails. The varnish was applied under normal conditions of use (namely one application every three days, after having removed the previous varnish).

Comparative composition 5 contained 0.05% of hexane, comparative composition 6 contained 0.05% by weight of citral, comparative composition 9 contained 0.05% by weight of caproic acid and composition 7 according to the present invention contained 0.05% by weight of sorbic acid. Varnish composition 8 did not contain any hardening agent (reference composition).

The respective compositions and also the results of the physical tests and observed by the panelists are collated in table 2 hereinafter.

TABLE 2

| | Comp. | Comp. | | Ex. 8 | Comp. |
|---|---|---|---|---|---|
| | ex. 5 | ex. 6 | Ex. 7 | (ref.) | ex. 9 |
| Compositions | | | | | |
| Butyl acetate | 42.53 | 42.52 | 42.52 | 42.57 | 42.52 |
| Ethyl acetate | 20.6 | 20.6 | 20.6 | 20.6 | 20.6 |
| Nitrocellulose | 14 | 14 | 14 | 14 | 14 |
| Polyester resin | 9.5 | 9.5 | 9.5 | 9.5 | 9.5 |
| Tributyl | 6 | 6 | 6 | 6 | 6 |
| acetylcitrate | | | | | |
| Isopropyl | 6 | 6 | 6 | 6 | 6 |
| alcohol | | | | | |
| Stearalkonium | 1.3 | 1.3 | 1.3 | 1.3 | 1.3 |
| bentonite | | | | | |
| Phosphoric | 0.03 | 0.03 | 0.03 | 0.03 | 0.03 |
| acid | | | | | |
| Hexanal | 0.05 | — | — | — | — |
| Citral | — | 0.05 | — | — | — |
| Sorbic acid | — | — | 0.05 | — | — |
| Caproic acid | | | | | 0.05 |
| Test results | | | | | |
| Gloss | 86.7 | 87.1 | 87 | 86.9 | |
| Flexibility | 4.5 | 4.4 | 4.6 | 4.4 | 4.4 |
| Varnish | 236 | 233 | 240 | 238 | 235 |
| hardness (s) | | | | | |
| Adhesion | 0 | 0 | 0 | 0 | 0 |
| Solids content | 28.1 | 28.3 | 27.9 | 28 | 28.1 |
| Stability | OK | OK | OK | Reference | OK |
| Odor | Strong | Light and | Absence | Reference | Absence |
| | | pleasant | | | |
| Nail hardness | | | | | |
| Number of | 23 | 18 | 25 | 12 | 14 |
| people | | | | | |
| answering Yes | | | | | |
| Percentage | 76.7 | 60.0 | 83.3 | 40.0 | 46.7 |

No clinical signs of intolerance were noted or felt by the persons tested. No symptoms of allergies were recorded.

In addition, more than 83% of the subjects found an overall improvement in the quality and the hardness of their nails. These results demonstrate the in vivo nail-hardening properties of sorbic acid, this occurring at a low concentration (0.05% by weight) in the varnish composition.

### Examples 11 and 12-Comparative Examples 10 and 13

### Sedimentation Stability and Color Fastness

To check the sedimentation stability, two coloring substances were added to the base varnish composition as claimed in claim 2:

firstly, a pearlescent agent: titanium oxide-coated mica sold under the name Flamenco Gold, 6-48 μm, by BASF,

and, secondly, Pigment Red 34: organic pigment sold by Sun Chemical.

This pigment was ground in order to be able to be incorporated. The grinding procedure included mixing the solvents with the pigment, stirring to deagglomerate the pigment, then incorporating the nitrocellulose. Once the latter had dissolved, the plasticizer was incorporated with stirring, which was continued for 20 minutes. The mixture obtained was then ground in a ball mill.

The Pigment Red 34 was thus ground with the following constituents (% by weight):

TABLE 3

| | |
|---|---|
| Butyl acetate | 30 |
| Ethyl acetate | 30 |
| Plasticizer (TAC) | 10 |
| Red 34 | 10 |
| Nitrocellulose | 20 |

The following shade was produced in each of the tests described hereinafter:

| | |
|---|---|
| Base varnish composition | 95% by weight |
| Solution containing the Red 34 | 3% by weight |

Titanium oxide-coated mica (Flamenco Gold, 6-48 μm, from BASF) 2% by weight The stability of the compositions thus prepared was evaluated by visual observation after storage for one month at 45° C.

TABLE 4

| | | Ex. 10 | | | Ex. 13 |
|---|---|---|---|---|---|
| | Ex. 2 | (comp.) | Ex. 11 | Ex. 12 | (comp.) |
| Composition | | | | | |
| Butyl acetate | 42.47 | 42.52 | 42.49 | 42.45 | 42.4 |
| Ethyl acetate | 20.6 | 20.6 | 20.6 | 20.6 | 20.6 |
| Nitrocellulose | 14 | 14 | 14 | 14 | 14 |
| Polyester resin | 9.5 | 9.5 | 9.5 | 9.5 | 9.5 |
| Tributyl | 6 | 6 | 6 | 6 | 6 |
| acetylcitrate | | | | | |
| Isopropyl | 6 | 6 | 6 | 6 | 6 |
| alcohol | | | | | |
| Stearalkonium | 1.3 | 1.3 | 1.3 | 1.3 | 1.3 |
| bentonite | | | | | |
| Phosphoric | 0.03 | | 0.01 | 0.05 | 0.1 |
| acid | | | | | |
| Citric acid | | 0.03 | | | |
| Sorbic acid | 0.1 | 0.1 | 0.1 | 0.1 | 0.1 |
| Results | | | | | |
| Yellowing | OK | OK | OK | OK | OK |
| stability | | | | | |
| Varnish | 248 | 242 | 240 | 240 | 237 |
| hardness (s) | | | | | |
| Viscosity | | | | | |
| (mPa · s) | | | | | |
| 6 rpm | 3250 | 1400 | 1890 | 4090 | 5630 |
| 60 rpm | 1020 | 980 | 1010 | 1080 | 1120 |
| 6 rpm | 2090 | 1230 | 1460 | 2530 | 3250 |
| Adhesion | 0 | 0 | 0 | 0 | 0 |
| Stability of | Stable | Sedimentation | Stable | Stable | Discolora- |
| the shade | | of the | | | tion of |
| described | | pearlescent | | | the shade |
| hereinabove | | agent | | | |

Conclusions:

Sedimentation is observed in the presence of citric acid in place of phosphoric acid. Phosphoric acid at 0.1% by weight in the varnish discolors the Pigment Red 34.

1. A non-aqueous thixotropic cosmetic nail varnish composition comprising:
a film-forming agent having
nitrocellulose,
at least one organic solvent, and
a thixotropic agent,
wherein said composition contains sorbic acid or one of its salts at a concentration of less than or equal to 1% by weight and in that the thixotropic agent, present at a concentration of between 0.1% and 4% by weight of the composition, is a clay modified with a quaternary amine, neutralized by a strong acid at a concentration of less than 0.1% by weight of the composition.

2. The cosmetic nail varnish composition as claimed in claim 1, wherein said cosmetic nail varnish contains a concentration of sorbic acid or one of its salts of less than or equal to 0.5% by weight.

3. The cosmetic nail varnish composition as claimed in claim 1, wherein the thixotropic agent is a clay modified with a quaternary amine, the clay being chosen from bentonite or hectorite.

4. The cosmetic nail varnish composition as claimed in claim 1, wherein the concentration of thixotropic agent is between 0.5% and 3% by weight, of the composition.

5. The cosmetic nail varnish composition as claimed in claim 1, wherein the strong acid which neutralizes the clay modified with a quaternary amine is phosphoric acid, preferably at a concentration of less than or equal to 0.05% by weight.

6. The cosmetic nail varnish composition as claimed in claim 1, wherein the concentration of nitrocellulose is between 8% and 30% by weight, preferably between 10% and 25% by weight.

7. The cosmetic nail varnish composition as claimed in claim 1, wherein the sorbic acid or one of its salts is used as a hardening agent for the nails.

8. The cosmetic nail varnish composition as claimed in claim 1, wherein the composition is a cosmetic composition in the form of a colored or colorless nail varnish, of a varnish base coat, or of a peelable varnish.

9. A sorbic acid, or one of its salts, as a nail-hardening agent at a concentration of less than 1% by weight,
Wherein said sorbic acid is included in a cosmetic composition as claimed in claim 1 that is applied to nails free from fungal infection.

10. The sorbic acid or one of its salts as claimed in claim 9, wherein the cosmetic composition contains a concentration of sorbic acid or of one of its salts of less than or equal to 0.5% by weight.

11. The sorbic acid or one of its salts as claimed in either of claim 9, wherein the cosmetic composition contains a concentration of sorbic acid or of one of its salts of between 0.01% and 0.5% by weight, more preferably of between 0.05% and 0.1% by weight.

12. A method for strengthening and hardening nails free from fungal infection, in particular cracked, thin, brittle, soft or split nails, consisting of:
topically applying to said nails a cosmetic varnish composition as claimed in claim 1.

13. The cosmetic nail varnish composition as claimed in claim 2, wherein said cosmetic nail varnish contains a concentration of sorbic acid or one of its salts between 0.01% at 0.5% by weight.

14. The cosmetic nail varnish composition as claimed in claim 2, wherein said cosmetic nail varnish contains a concentration of sorbic acid or one of its salts between 0.05% and 0.1% by weight.

15. The cosmetic nail varnish composition as claimed in claim 4, wherein the concentration of thixotropic agent is between 1% and 2% by weight, of the composition.

16. The cosmetic nail varnish composition as claimed in claim 5, wherein the strong acid which neutralizes the clay modified with a quaternary amine is phosphoric acid, preferably at a concentration of less than or equal to 0.03% by weight, of the composition.

17. The cosmetic nail varnish composition as claimed in claim 7, wherein the concentration of nitrocellulose is between 10% and 25% by weight, of the composition.

18. The cosmetic nail varnish composition as claimed in claim 7, wherein the concentration of nitrocellulose is between 12% and 20% by weight, of the composition.

19. The sorbic acid or one of its salts as claimed in either of claim 9, wherein the cosmetic composition contains a concentration of sorbic acid or of one of its salts of between 0.05% and 0.1% by weight.
